(19) United States
(12) Patent Application Publication
(10) Pub. No.: US 2011/0270145 A1
(43) Pub. Date: Nov. 3, 2011

(54) Hand-Arm Stabilizer For Immobilizing A Person's Wrist While Allowing That Person To Hold And Operate A Hand Held Device

(76) Inventor: Mark Nail, Wrightsville, PA (US)

(21) Appl. No.: 12/769,835

(22) Filed: Apr. 29, 2010

(51) Int. Cl. A61F 5/00

(52) U.S. Cl. 602/21

(57) ABSTRACT

Apparatus and method of immobilizing a person's wrist comprising a first elongated member having a first end coupled to a butt end and a second end and a second elongated member having a first end slidably or pivotally coupled to the second end of the first member and a second end. A flat yieldable member is attached to the second end of the second member and a yieldable pad having an obverse side is attached to the flat yieldable member and a reverse side adapted to be placed against the back of a person's hand. First and second straps are attached to the yieldable pad for attaching the yieldable pad and flat yieldable member to a person's hand.

### BACKGROUND OF THE INVENTION

1. Field of the Invention

The present invention relates to apparatus and method for operating a hand held device at arm's length and more specifically to a telescoping and/or folding apparatus which, when attached to a person's hand and arm on each side of the person's wrist and positioned against the person's shoulder, stabilizes that person's wrist and elbow to allow a user of the apparatus to hold and operate a hand held device at arms length regardless of the purpose or type of device that is being held by the person and where the wrist elbow stabilizer is not attached to or is a part of the hand held device. In one embodiment the hand held device can be a camera. In another embodiment the hand held device can be a hand gun.

2. Description of Related Art

Apparatus and methods for providing shoulder, telescoping and/or folding shoulder stock for a gun is known in the prior art. More specifically, by way of example, U.S. PreGrant Publication No. 2007/0056202 to Oz discloses a telescoping stock for a firearm where the stock includes a stock body having an upper mounting rail, a buffer tube for receiving a firearm buffer, the buffer tube has a complementary mounting rail, and locking means for selectably locking the buffer tube in any one of a plurality of positions relative to the stock body.

U.S. Pat. No. 7,552,557 to Mabry discloses a pivotable shoulder stock for use in combination with a handgun having a mirror and allows the user to aim and fire an equipped laser handgun around the corner of a building or other obstacle. The user is able to fire with relative accuracy from behind a building or other obstacle using the mirror attached to the shoulder stock. The mirror can be adjustably positioned for viewing in order to fire the handgun at about a ninety degree angle in either a clockwise or counterclockwise direction. The mirror can be revolved to a downward posture when firing the handgun in a linear direction similar to a rifle or for storage purposes. The pivotable shoulder stock can be quickly adjusted by latching the second section against the first section for use as a hand weapon rather than being shoulder fired.

U.S. Pat. No. 6,662,485 to Kay discloses a firearm interface for a butt stock and pistol grip that provides a means to attach a pistol grip or a butt stock assembly to a shotgun. It can be used for either modality individually or both in combination. It allows the utilization of an M16/M4 style pistol grip and the adjustable, as well as the fixed butt stocks of M4/M16 style rifles and carbines. By facilitating the use of M4/M16 style receiver extensions and M16/M4 style pistol grips the butt stock and pistol grip interface brings a vast array of options to the firearms to which it is affixed.

U.S. Pat. No. 6,651,371 to Fitzpatrick, et al. discloses a modular stock system for rifles. The system replaces the rifle's buffer tube with a modified one containing a mount for a constant cheek weld and a rail track for adjustment. A hind portion then mounts on the replacement buffer tube. The hind portion is interchangeable depending on the user's needs. Various configurations of the hind portion are discussed, including adjustable stocks, fixed stocks, and specialized stocks.

U.S. Pat. No. 6,374,528 to Davis, et al. discloses an adaptor kit for mounting a stock on a firearm receiver having any one of a plurality of different configurations. The kit includes one or both of a rear stock and a fore-stock and a set of front and/or rear adaptors that enable the rear and/or fore-stock to be interchangeably mounted on differently configured firearm receivers so that the mounted stock and receiver meets predetermined design parameters including finger pull, shoulder pull and drop parameters.

U.S. Pat. No. 6,560,911 to Sharp discloses an adjustable-length stock for a shoulder fired weapon, which includes an extendible shoulder piece installed on a shortened gun stock. The extendible shoulder piece includes a butt plate for placement against the shoulder of a user, and a telescoping connection between the butt plate and the shortened gun stock. The connection may have at least one support rod and two support rods telescopically sliding within support tubes. After adjustment, the stock is locked at the selected length, to hold a desired length. The two connections between the butt plate and the gun stock are vertically-distanced as much as possible to stabilize the butt plate.

U.S. Pat. No. 4,512,10 to Waterman, Jr. discloses a butt stock for a rifle which has a lower receiver extension with a shell of high impact plastic and a molded foam core which defines a stowage compartment. The shell includes a longitudinally extending web of semi-circular cross-section which, together with the inner periphery of the upper portion shell, defines a cylindrical volume for receiving the receiver extension. The web serves to more evenly distribute impact loads to the lower receiver of the rifle when the rifle is impacted through the butt stock and generally strengthens the butt stock and facilitates the flow of core material during the molding process.

U.S. Pat. No. 4,312,146 to Koon, Jr. discloses a rifle having a stock, a receiver, a barrel, a trigger assembly, a magazine, and stock inserts forming mounting blocks for supporting the receiver in the stock. The magazine is a removable integral sheet metal member with inwardly turned longitudinal retainer flanges along the top rear edges of the side walls, a vertical internal retainer boss along each side wall spaced forwardly from the retainer flanges, cartridge case loading ramp edge surfaces along the top edge of each of the side walls of the magazine spaced from the longitudinal retainer flanges, and internal stops on the side walls for limiting the upward movement of a follower within the magazine.

U.S. Pat. No. 3,798,818 to Casull discloses an automatic hand gun which is attachable to and detachable from an auxiliary stock to convert the gun to a shoulder gun when desired by means of cooperating quick detachable coupling means, one of which is located on the bottom of a cartridge clip and the other of which is located at or on the end of the auxiliary stock.

U.S. Pat. No. 3,685,194 to Coon discloses an assembly for converting a handgun to a rifle where an elongated rifle barrel has locking means which is identical to the locking means on the handgun barrel is substituted within the handgun in place of the conventional short handgun barrel. The handgrip cover plates are removed from the handgun and the handgun with the rifle barrel is placed between a pair of handgrip engaging plates which form a portion of a rifle stock. The handgrip cover plates are placed on the outer faces of the handgrip engaging plates and a pair of securing members are inserted through aligned apertures in the handgrip cover plates, the handgrip engaging plates and the handgrip to secure the handgun within the rifle stock.

U.S. Pat. No. 6,318,014 to Porter discloses an adapter for converting a handgun into a rifle or carbine. The adapter includes an elongated member having a stock and a pair of sidewalls forming a cavity. A pair of guide rails is positioned on an inner edge of the cavity, and a locking bar is mounted within the sidewalls and extends across the cavity. The locking bar is selectively positioned between a locked position and an unlocked position. A handgun mounts into the adapter by aligning channels on the outer edge of the handgun into the guide rails. The handgun is slid along the guide rails and into the locking mechanism for attachment.

### SUMMARY OF THE INVENTION

In an exemplary embodiment of the present invention, there is disclosed a hand arm stabilizer for immobilizing a person's wrist comprising:
a first elongated member having a first end coupled to a butt end and a second end;
a second elongated member having a first end slidably coupled to the second end of the first member and a second end;
a flat yieldable member attached to the second end of said second member;
a yieldable pad having an obverse side attached to the flat yieldable member and a reverse side adapted to be placed against the back of a person's hand; and
first and second straps attached to the yieldable pad for attaching the yieldable pad and flat yieldable member to a person's hand.

In another exemplary embodiment of the present invention, there is disclosed a hand arm stabilizer for immobilizing a person's wrist comprising:
a first elongated member having a first end coupled to a butt end and a second end;
a second elongated member having a first end pivotally coupled to the second end of the first member and a second end;
a flat yieldable member attached to the second end of said second member;
a yieldable pad having an obverse side attached to the flat yieldable member and a reverse side adapted to be placed against the back of a person's hand; and
first and second straps attached to the yieldable pad for attaching the yieldable pad and flat yieldable member to a person's hand.

In another exemplary embodiment of the present invention, there is disclosed a method of immobilizing a person's wrist comprises:
providing a first elongated member having a first end coupled to a butt end and a second end;
providing a second elongated member having a first end slidably or pivotally coupled to the second end of the first member and a second end;
attaching a flat yieldable member to the second end of said second member;
providing a yieldable pad having an obverse side attached to the flat yieldable member and a reverse side adapted to be placed against the back of a person's hand; and
attaching first and second straps to the yieldable pad for attaching the yieldable pad and flat yieldable member to a person's hand.

The more important aspects and features of the invention have thus been outlined in order that the more detailed description that follows may be better understood and in order that the present contribution to the art may better be appreciated. Additional features of the invention will be described hereinafter and will form the subject matter of the claims that follow.

Many aspects and features of this invention will appear from the following description and appended claims, reference being made to the accompanying drawings which form a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a side view of a wrist arm telescoping stabilizer which can be used to provide increased support for a hand held device which is grasped by the person's hand where the hand held device can be a camera or a hand held gun; and

FIG. 2 is a side view of a wrist arm folding stabilizer which can be used to provide increased support for a hand held device which is grasped by the person's hand where the hand held device can be a camera or a hand held gun.

### DESCRIPTION OF THE PREFERRED EMBODIMENT

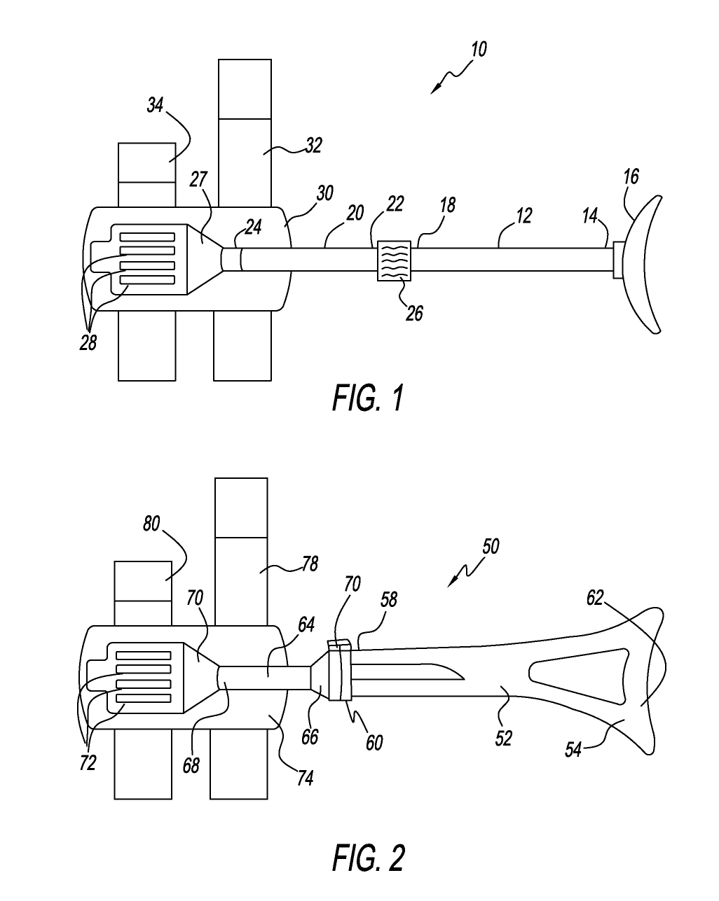

Referring to FIG. 1, there is shown a side view of a wrist-arm telescoping stabilizer which can be used to provide increased support for a hand held device that is grasped by a the user's hand where the hand held device can be a camera or a hand held gun. Stabilizer 10 includes a first member 12 which can be a tube made of aluminium or any other light weight material such as fibreglass having an outside diameter of one inch, more or less, a butt end 14 and a telescoping end 18. The butt end 14 is attached to a shoulder engaging member 16 which can be made of aluminium, fibreglass, wood, etc. and, if desired, can be covered with a padding of any desired material such as foam rubber or a shock absorbing material such as a gel located in a pliable plastic envelope. The telescoping end of the first member 12 may be have a loop or hook which is adapted to be connected to a shoulder strap which, when placed around a user's shoulder will provide additional support to the user's arm while holding a camera or a gun. In addition to, or in place of the shoulder strap, a mono pod may be located under the user's arm or under the first member to provide additional support while the user is holding a camera or a gun. The actual configuration of the shoulder engaging member can replicate the butt end of any one of the many rifle shoulder stocks that are currently being used with rifles on today's market. The first member 12, including the shoulder engaging member 16 can have a total length of about thirteen inches, more or less.

The telescoping end 18 of tube 12 is slidably connected to a second tubular member 20 having a first end 22 and a second end 24. Tube 20 can be made of aluminium or any other light weight material such as fibreglass and has an inside diameter that is slightly larger than the outside diameter of first member 12 to allow tube 12 to freely slide in and out of tube 20. A twist locking collar 26 which is rotatably coupled to end 22 of tube 20 is provided to lock tube 12 to tube 20. Tube 20 can have a length of about twelve inches, more or less and tube 20 can have a length of thirteen inches, more or less. The end 24 of tube 20 is attached to a flat yieldable “back of the hand” attachment member 27 which has a length of about five inches, more or less. Member 27 can be made of a sheet of aluminium or fibreglass which can have spaced apart elongated members 28 which allows it to flex along the transverse axis of tube 20 but not along the longitudinal axis of tube 20. This flexing action allows member 27 to be comfortably located on the back of a person's hand while locking the person's wrist to one position. A soft but sturdy fabric pad 30 is securely attached to member 27 and to the end 24 of tube 20 with an adhesive such as epoxy or the like. Pad 30 can have a length of about eight inches, more or less and a width of about three inches, more or less. A first attachment strap 32 is attached to the rear of pad 30 by sewing or with an adhesive, and a second attachment strap 34 is attached to the front of pad 30 by sewing or with an adhesive. Attachment strap 32 can have a width of about two inches, more or less, and can have a hook and loop attachment means such as a Velcro with hooks are on one side of the strap and loops on the other side of the strap. In a similar arrangement, attachment strap 34 can have a width of about one inch, more or less, and can have a hook and loop attachment means such as Velcro with hooks on one side of the strap and loops on the other side of the strap.

In FIG. 1 the obverse side of pad 30 is shown. The reverse side of pad 30 is not visible. In use, the reverse side of pad 30 of wrist-arm telescoping stabilizer 10 is placed against the back of a person's hand and strap 32 is securely wrapped around the user's arm above the wrist. Then strap 34 is securely wrapped around the user's hand between his/her thumb and forefinger. At this time the user's wrist motion is substantially frozen and his/her hand is in a fixed position relative to his/her arm. However, the user can still bend his/her elbow and fingers. Now the user places shoulder engaging member 16 against his/her shoulder where his/her arm meets his/her body and slides tube 12 either in or out of tube 20 until his/her elbow is straight out or slightly bent. The user now turns twist lock 26 to lock tube 12 to tube 20. At this time the wrist-arm stabilizer 10 follows a path that goes from inside the user's arm where his/her arm meets his/her body, over his/her elbow to the back of his/her hand to substantially stabilize and lock the user's hand to his/her arm. It is to be noted that the user can still move his/her hand left, right, up and down about his/her shoulder joint and can freely move his/her thumb and fingers to hold and operate a camera or a hand held gun. In this embodiment the total length of wrist arm stabilizer is about thirty inches. It is understood that the length of the wrist-arm stabilizer can be less than or greater than thirty inches by sliding tube 12 into tube 20 until the desired length is obtained.

In another embodiment and referring to FIG. 2, there is shown a side view of a wrist-arm folding stabilizer which can be used to provide increased support for a hand held device which is grasped by the person's hand where the hand held device can be a hand held gun.

Stabilizer 50 includes a first member 52 which can be a flat member made of aluminium or any other light weight material such as fibreglass having a width of about one inch, more or less, a thickness of about three-eighths of an inch more or less, a butt end 54 and a folding end 58 securely attached to a hinge 60. The butt end 54 is attached to a shoulder engaging member 62 which can be made of aluminium, fibreglass, wood, etc. and, if desired, can be covered with a padding of any desired material such as foam rubber or a shock absorbing material such as a gel located in a pliable plastic envelope. The folding end of the first member 52 may be have a loop or hook which is adapted to be connected to a shoulder strap which, when placed around a user's shoulder will provide additional support to the user's arm while holding a camera or a gun. In addition to, or in place of the shoulder strap, a mono pod may be located under the user's arm or under the first member to provide additional support while the user is holding a camera or a gun. The actual configuration of the shoulder engaging member can replicate the butt end of any one of the many rifle shoulder stocks currently being used with rifles on today's market. The first member 52, including the shoulder engaging member 62 can have a total length of eight inches, more or less.

The hinge end 58 of first member 52 is pivotally connected to a second member 64 having a first end 66 which is attached to the hinge 60 and a second end 68. Member 64 can be made of aluminium or any other light weight material such as fibreglass and can be a flat member made of aluminium or any other light weight material such as fibreglass having a width of about one inch, more or less and a thickness of about three-eighths of an inch. A locking mechanism 70 is coupled to hinge 60 to lock first member 52 to second member 64. Member 52 can have a length of about eight inches, more or less and member 64 can have a length of about five inches, more or less. The end 68 of member 64 is attached to a flat yieldable “back of the hand” member 70 which has a length of about five inches, more or less. Member 70 can be made of a sheet of aluminium or fibreglass which can have spaced apart elongated members 72 which allows it to flex across the transverse axis of member 64 but not along the longitudinal axis of member 64. This flexing action allows member 70 to be comfortably located on the back of a person's hand while substantially locking the person's wrist to one position. A soft but sturdy fabric pad 74 is securely attached to member 70 and to the end 68 of member 64 with an adhesive such as epoxy or the like. Pad 74 can have a length of about eight inches, more or less, and a width of about three inches, more or less. A first attachment strap 78 is attached to the rear of pad 74 by sewing or with an adhesive, and a second attachment strap 80 is attached to the front of pad 74 by sewing or with an adhesive. Attachment strap 78 can have a width of about two inches, more or less, and can have a hook and loop attachment means such as Velcro with hooks on one side of the strap and loops on the other side of the strap. In a similar arrangement, attachment strap 80 can have a width of about one inch, more or less, and can have a hook and loop attachment means such as Velcro with hooks on one side of the strap and loops on the other side of the strap.

In FIG. 2 the obverse side of pad 74 is shown. The reverse side of pad 74 is not visible. In use, the reverse side of pad 74 of wrist-arm folding stabilizer 50 is placed against the back of a person's hand and strap 78 is securely wrapped around the user's arm above the wrist. Then strap 80 is securely wrapped around the user's hand between his/her thumb and forefinger. At this time the user's wrist is substantially frozen and his/her hand is in a fixed position relative to his/her lower arm. However, the user can still bend his/her elbow and fingers. Now the user places shoulder engaging member 62 of member 52, which is aligned with member 64 and with hinge 60 locked, against his/her shoulder where his/her arm meets his/her body. At this time the wrist-arm stabilizer 50 traces a path that goes from inside the user's arm where his/her arm meets his/her body, and over his/her elbow to the back of his/her hand to substantially stabilize and lock the user's hand to his/her arm. It is to be noted that the user can still move his/her hand left, right, up and down about his/her shoulder joint and can freely move his/her thumb and fingers to hold and operate a camera or a hand held gun. In this embodiment the total length of wrist-arm stabilizer is about eighteen inches. It is understood that the length of the wrist-arm stabilizer can be less than of greater than eighteen inches.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A hand arm stabilizer for immobilizing a person's wrist comprising:
a first elongated member having a first end coupled to a butt end and a second end;
a second elongated member having a first end slidably coupled to the second end of the first member and a second end;
a flat yieldable member attached to the second end of said second member;
a yieldable pad having an obverse side attached to the flat yieldable member and a reverse side adapted to be placed against the back of a person's hand; and
first and second straps attached to the yieldable pad for attaching the yieldable pad and flat yieldable member to a person's hand.

2. The hand arm stabilizer of claim 1 wherein the first attachment strap is adapted to be wrapped around a person's arm above the wrist.

3. The hand arm stabilizer of claim 2 wherein the second attachment strap is adapted to be wrapped around a person's palm between the thumb and fore finger.

4. The hand arm stabilizer of claim 3 wherein the flat yieldable member can flex along an axis that is transverse to the longitudinal axis of the second elongated member.

5. The hand arm stabilizer of claim 4 wherein the second elongated member is a tube and the first elongated member is a tube which telescopes into the second tube.

6. The hand arm stabilizer of claim 5 wherein locking means is coupled to lock the first elongated member to the second elongated member.

7. The hand arm stabilizer of claim 6 wherein the locking means is a twist lock.

8. The hand arm stabilizer of claim 7 wherein the first and second elongated members or made of aluminum or fiberglass.

9. A hand arm stabilizer for immobilizing a persons wrist comprising:
a first elongated member having a first end coupled to a butt end and a second end;
a second elongated member having a first end pivotally coupled to the second end of the first member and a second end;
a flat yieldable member attached to the second end of said second member;
a yieldable pad having an obverse side attached to the flat yieldable member and a reverse side adapted to be placed against the back of a person's hand; and
first and second straps attached to the yieldable pad for attaching the yieldable pad and flat yieldable member to a person's hand.

10. The hand arm stabilizer of claim 9 wherein the first attachment strap is adapted to be wrapped around a person's arm above the wrist.

11. The hand arm stabilizer of claim 10 wherein the second attachment strap is adapted to be wrapped around a person's palm between the thumb and fore finger.

12. The hand arm stabilizer of claim 11 wherein the flat yieldable member can flex along an axis that is transverse to the longitudinal axis of the second elongated member.

13. The hand arm stabilizer of claim 12 wherein the second elongated member has a rectangular or square cross section and the first elongated member has a rectangular or square cross section which is coupled with a hinge to the second member.

14. The hand arm stabilizer of claim 13 wherein locking means is coupled to lock the hinge to longitudinally align the first elongated member with the second elongated member.

15. The hand arm stabilizer of claim 14 wherein the first and second straps have hooks one side that engage loops on the other side.

16. The hand arm stabilizer of claim 15 wherein the first and second elongated members or made of aluminum or fiberglass.

17. A method of immobilizing a persons wrist comprises:
providing a first elongated member having a first end coupled to a butt end and a second end;
providing a second elongated member having a first end slidably or pivotally coupled to the second end of the first member and a second end;
attaching a flat yieldable member to the second end of said second member;
providing a yieldable pad having an obverse side attached to the flat yieldable member and a reverse side adapted to be placed against the back of a person's hand; and
attaching first and second straps to the yieldable pad for attaching the yieldable pad and flat yieldable member to a person's hand.

18. The method of claim 17 wherein the first attachment strap is adapted to be wrapped around a person's arm above the wrist.

19. The method of claim 18 wherein the second attachment strap is adapted to be wrapped around a person's palm between the thumb and fore finger.
